(19) United States
(12) Patent Application Publication
(10) Pub. No.: US 2023/0000433 A1
(43) Pub. Date: Jan. 5, 2023

(54) INTRAORAL DEVICE

(71) Applicant: Oxford University Innovation Limited, Oxford (GB)

(72) Inventors: Jeroen Henricus Maria BERGMANN, Oxford (GB); Man Ting KWONG, Oxford (GB); Leonardo DE ALMEIDA E BUENO, Oxford (GB); William MILNTHORPE, Oxford (GB); Runbei CHENG, Oxford (GB)

(21) Appl. No.: 17/781,738

(22) PCT Filed: Dec. 3, 2020
(86) PCT No.: PCT/GB2020/053095

§ 371 Date: Jun. 2, 2022

(30) Foreign Application Priority Data
Dec. 5, 2019 (GB) 1917818.5

(51) Int. Cl. A61B 5/00; A61B 7/04; A61B 7/00; A61B 5/08

(52) U.S. Cl. CPC A61B 5/682; A61B 7/04; A61B 5/0002; A61B 7/003; A61B 5/0816; A61B 5/0803; A61B 5/725; A61B 2503/10

(57) ABSTRACT

There is provided a device for measuring fatigue of a person, the device comprising a frame configured to be worn within the mouth of the person, a microphone mounted within the frame and configured to measure sound data, and a cavity located within the frame and adjacent to the microphone, wherein the cavity does not communicate with the environment surrounding the frame. There is also provided a computer-implemented method for determining a fatigue metric representing a level of physical fatigue of a person

The present invention relates to a device for monitoring the health of a person. In particular, the present invention relates to an intraoral device for measuring a person's breathing and changes to the person's breathing to monitor fatigue.

Physical exercise and training are important in maintaining a person's health and improving and maintaining performance for athletes and other sportspeople. However, overexertion can reduce the effectiveness of training, and lead to increased risk of injury. Therefore, effective surveillance of exercise and training routines is essential to develop healthy athletic profiles while minimising the risk of injury. This also allows the assessment of the effectiveness of training programs.

To achieve this, it is important to be able to monitor the level of fatigue in sportspeople during training. However, it can be difficult for sportspeople to assess their own fatigue accurately during physical activity, and the symptoms of overexertion are often visible to observers only once they become extreme and potentially dangerous. Existing methods of measuring fatigue often involve bulky equipment such as breathing tubes, and are unsuitable for use in real-world training environments.

Accordingly, there is a need to be able to monitor fatigue in sportspeople during physical activity in an accurate and unobtrusive manner. It is an aim of the invention to provide a device which at least partially addresses the above problems.

According to a first aspect of the invention, there is provided a device for measuring fatigue of a person, the device comprising a frame configured to be worn within the mouth of the person, a microphone mounted within the frame and configured to measure sound data, and a cavity located within the frame and adjacent to the microphone, wherein the cavity does not communicate with the environment surrounding the frame.

By collecting sound data within a person's mouth, information about their breathing can be collected and used to monitor fatigue. Providing a cavity within the frame adjacent to the microphone allows the microphone to perform effectively even when it is encapsulated within the device. This means reliable data can be measured while protecting the microphone from the environment of the person's mouth.

In an embodiment, a first surface of the frame is configured to engage against the mouth and/or teeth when the frame is worn within the mouth of the person, and the cavity is located between the microphone and a second surface of the frame that does not engage against the mouth and/or teeth when the frame is worn within the mouth of the person. By locating the cavity between the microphone and an exposed surface of the frame, the cavity can more effectively transmit sound to the microphone.

In an embodiment, the microphone is substantially parallel to a surface of the frame at a point on the surface of the frame closest to the microphone. Orienting the microphone parallel to the surface of the frame allows sound to more directly be transmitted to the microphone from the exterior of the frame. It also simplifies the manufacture of the device where the frame is formed from laminated layers of material.

In an embodiment, the microphone is mounted within a portion of the frame that is within the oral cavity of the person when the frame is worn within the mouth of the person. This ensures that the microphone is positioned within a portion of the frame that is more directly exposed to air flowing in and out of a person's mouth than a portion that is, for example, between the teeth and the cheeks or lips. Therefore, vibrations caused by the air flowing in and out of the mouth are more effectively transmitted to the microphone.

In an embodiment, the microphone is positioned within a portion of the frame that engages with the roof of the mouth of the person when the frame is worn within the mouth of the person. Placing the microphone at the roof of the mouth positions the microphone away from the teeth of the person, preventing the microphone and associated wiring from interfering with any protective function that may be provided by the frame.

In an embodiment, the cavity has a minimum dimension of at least 0.05 mm. Too small a cavity would not as effectively allow the microphone to operate when encapsulated within the frame. Requiring a minimum size of cavity ensures a minimum level of signal quality.

In an embodiment, the cavity has a maximum dimension of at most 5 mm. Too large a cavity would compromise the structural properties of the device, and increase the likelihood of damage occurring during use.

In an embodiment, a minimum distance between the microphone and an edge of the cavity is at most 0.1 mm. Ensuring the cavity is proximate to the microphone increases the effect of the cavity in improving sound data quality.

In an embodiment, the microphone is exposed to the cavity. Having the microphone exposed to the cavity is particularly advantageous, as it allows the microphone's active surface to oscillate more freely in response to received sound signals.

In an embodiment, a minimum distance between the microphone and a surface of the frame is at most 10 mm. Providing the microphone close to the surface of the frame reduces sound signal attenuation due to the material of the frame.

In an embodiment, the device further comprises a processor configured to receive sound data measured by the microphone, and determine a fatigue metric representing a level of physical fatigue of the person using the received sound data. A fatigue metric provides a way to assess the person's fatigue level, allowing decisions to be taken on how to respond to a change in fatigue level.

In an embodiment, the device further comprises a light emitter mounted within the frame and configured to emit light, wherein the processor is further configured to control emission of light by the light emitter based on the determined fatigue metric. Visual feedback provided by the light emitter can allow a change in fatigue level to be rapidly and easily detected by observers.

In an embodiment, the processor is configured to control the light emitter to emit light when the fatigue metric is above a predetermined threshold. This would allow the predetermined threshold to be set, for example, at a dangerous or potentially dangerous fatigue level, so that overexertion can be easily monitored by observers.

In an embodiment, the processor is configured to control the light emitter to emit light of a first colour when the fatigue metric is at or below a predetermined threshold, and to emit light of a second colour when the fatigue metric is above the predetermined threshold. Such a feature contributes to the improved monitoring of fatigue level by providing reassurance to observers that the person's fatigue metric is within a safe range when it is below the predetermined threshold.

In an embodiment, the device further comprises communication means mounted within the frame. This allows data about the person's fatigue levels to be transmitted by the device, for example to assess the fatigue metric in real-time during training.

In an embodiment, the communication means comprises a wireless communication means. This is particularly convenient for unobtrusive monitoring of fatigue levels by observers.

In an embodiment, the processor is separate from the frame, and the communication means is configured to transmit the sound data to the processor. This can reduce the complexity of the part of the device within the person's mouth, thereby allowing it to be made more compact.

In an embodiment, the processor is mounted within the frame. This can allow the processing of the fatigue metric to be carried out with a lower latency, and without requiring a separate external part of the device.

In an embodiment, a length of a wire carrying sound data between the microphone and the processor is less than 10 cm. Reducing the wire length reduces signal degradation that may be caused by interference in the wire.

In an embodiment, at least a portion of the frame is configured to engage with the teeth of the person. Engaging with the teeth enables the device to be easily retained within the person's mouth.

In an embodiment, the frame is configured to surround one or more of the teeth of the person. Surrounding the teeth allows the device to perform a protective function for the person's teeth, as well as monitoring their fatigue level.

In an embodiment, the frame is a mouthguard. Many sportspeople are familiar with wearing mouthguards, and embodying the device in a mouthguard allows it to perform protective and fatigue monitoring functions in an unobtrusive manner.

In an embodiment, the frame is formed from an energy absorbing material. Using energy absorbing materials improves the protective function of the mouthguard for the user.

In an embodiment, components of the device mounted within the frame are embedded within the energy absorbing material. Embedding the components within the frame protects the components that measure the sound data from mechanical shock and moisture.

In an embodiment, the device further comprises a memory module configured to store the measured sound data, and/or store information calculated using the measured sound data. Storing data within the device can allow fatigue levels during a training session to be recorded for later analysis.

According to a second aspect of the invention, there is provided a computer-implemented method for determining a fatigue metric representing a level of physical fatigue of a person, the method comprising receiving sound data measured within the mouth of the person, processing the sound data to determine a breathing rate of the person and an amplitude of the sound data, determining the fatigue metric using the breathing rate and the amplitude.

Thereby a method is provided that allows a measure of fatigue to be calculated, using data that can be measured during training without requiring complex or intrusive equipment to be worn by the person. Changes in breathing affect the perception of exertion, which is not the case with other measures affected by physical exertion such as heart rate. Using information derived from breathing therefore gives a more accurate identified of the perceived exertion level of the person. It can also provide insights into recovery after physical exertion has stopped or reduced in intensity.

In an embodiment, the fatigue metric is determined using a product of the breathing rate and the amplitude. This provides a fatigue metric which is robust, but nonetheless straightforward to calculate and so does not require high levels of processing power.

In an embodiment, the sound data, and/or the product of the breathing rate and the amplitude of the sound data is filtered. Filtering the sound data or the fatigue metric can make the determined fatigue metric more robust to transient fluctuations or noise in the sound data.

In an embodiment, processing the sound data comprises bandpass filtering the sound data to exclude frequencies outside of the range 10 Hz to 2 kHz. Bandpass filtering allows unwanted sound signals outside the desired frequency range to be excluded from contributing to the determination of the fatigue metric, thereby reducing noise and improving the accuracy of the fatigue metric.

In an embodiment, determining the breathing rate and the amplitude comprises calculating a moving average of the breathing rate and/or the amplitude over at least three breaths taken by the person. Using a moving average reduces the sensitivity of the fatigue metric to fluctuations in the sound data, and provides a smoother indication of the change in fatigue level.

In an embodiment, determining the fatigue metric comprises calculating an exponential moving average of the product of the breathing rate and the amplitude. This provides a weighted average that allows for more recent measurements to have higher weighting, while still taking account of older measurements, thereby stabilising the determined fatigue metric without too greatly affecting its ability to react to rapid changes in the person's behaviour.

Embodiments of the invention will now be described, by way of example only, with reference to the accompanying drawings in which corresponding reference symbols represent corresponding parts, and in which:

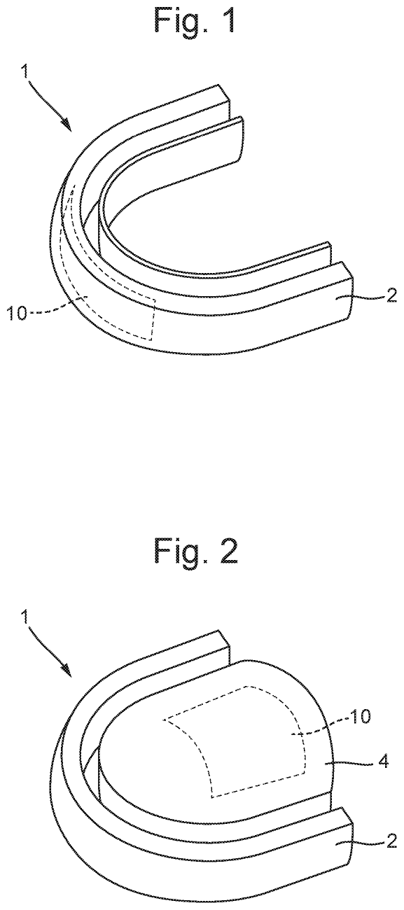

FIG. 1 shows an embodiment of a device according to the first aspect of the invention;

FIG. 2 shows a device with a portion that engages with the roof of a user's mouth;

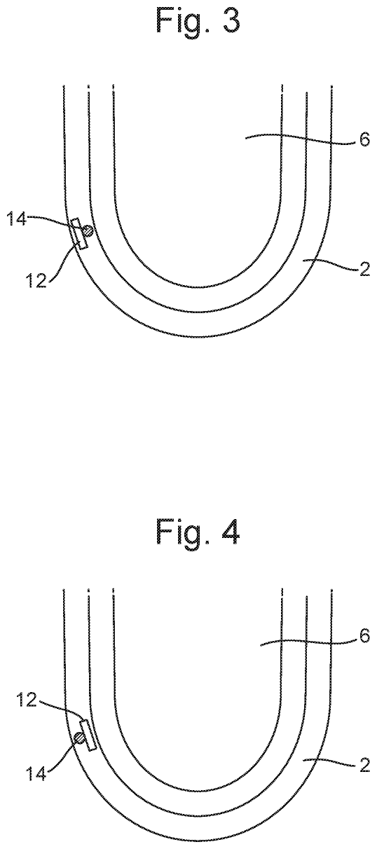

FIG. 3 is a sectional view of an embodiment, showing the microphone and cavity;

FIG. 4 shows an alternative arrangement of the microphone and cavity;

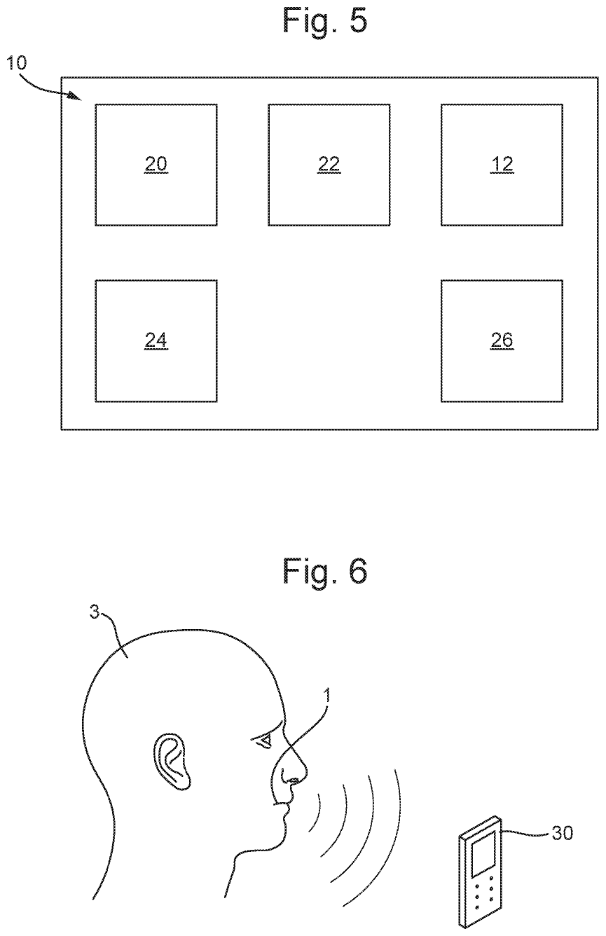

FIG. 5 is a schematic diagram of the electronic components of the device;

FIG. 6 shows an embodiment with a component of the device separate from the frame;

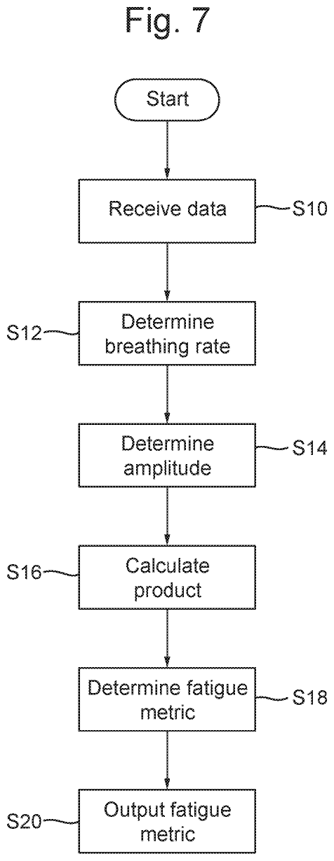

FIG. 7 is a flowchart of an embodiment of the method of the second aspect of the invention.

FIG. 1 shows a device 1 for measuring fatigue of a person 3 in accordance with an embodiment of the present invention. The device 1 comprises a frame 2 configured to be worn within the mouth of the person 3, for example when the person 3 is participating in physical activity such as a sports match or training session. In an embodiment, at least a portion of the frame 2 is configured to engage with the teeth 6 of the person 3, for example the frame 2 may be configured to surround one or more of the teeth 6 of the person 3. The frame 2 has a curved, U-shape to follow the teeth of the person 3. The frame 2 also has a curved, U-shaped cross-section to fit around the teeth 6 of the person 3. This is shown in further detail in FIGS. 3 and 4. Some or all of the electronic components of the device 1 may be mounted on a circuit board 10 within the frame 2. The circuit board 10 may be curved to follow the profile of the frame 2, as shown in FIG. 1, although this is not essential. The circuit board may be a printed circuit board (PCB) and may have a combination of rigid and flexible regions. In some embodiments, such as that shown in FIG. 2, the frame 2 may additionally comprise an arch portion 4 configured to fit into the roof of the person's mouth when worn. The arch portion 4 may be used to improve the ability of the device 1 to be retained within the mouth during physical activity, or may be used to house some or all of the electronic components of the device 1. For example, in FIG. 2 the circuit board 10 is located within the arch portion 4.

The frame 2 may be manufactured using injection moulding, 3D printing, vacuum forming, vacuum thermoforming, or any other suitable technique depending on the material from which the frame 2 is formed. In an embodiment, the frame 2 is a mouthguard. The frame 2 may be individually manufactured to fit the mouth of an individual person. For example, where the frame 2 is configured to engage with the teeth 6 of the individual person, the frame may be manufactured using a mould of the teeth of that individual person. In an embodiment, the frame 2 is formed from an energy absorbing material. The energy absorbing material can help to protect the teeth 6 of the person 3 from injury during the physical activity. The thickness of the frame 2 may be chosen to provide adequate protection for the person's teeth. The thickness may vary in different embodiments of the device 1 depending on the activity that will be carried out by the person while the device 1 is in use. For example, a greater thickness may be required for the device 1 in an embodiment designed for use while playing rugby than an embodiment designed for use during long-distance running. In an embodiment, components of the device 1 mounted within the frame 2 are embedded within the energy absorbing material. In such an embodiment, the energy absorbing material also functions to protect the components of the device 1. In an embodiment, the frame 2 is formed from dentistry approved mouthguard material. Examples of materials that may be used include Arnitel®, D30®, and ethylene-vinyl acetate (EVA). In an embodiment, the energy-absorbing material is ethylene-vinyl acetate (EVA). The choice of material may be affected by the manufacturing method used to manufacture the frame 2. For example, Arnitel® may be particularly suitable where 3D printing is used. In an embodiment, the frame 2 has a layered structure, for example formed from multiple sheets of EVA. The frame 2 may be also formed from a combination of different materials, such as multiple medical-grade thermo-polymers. For example, the frame 2 may comprise an external rubber frame with at least one EVA liner.

The device 1 further comprises a microphone 12 mounted within the frame and configured to measure sound data. In an embodiment, the microphone 12 is substantially parallel to a surface of the frame 2 at a point on the surface of the frame 2 closest to the microphone 12. In such an embodiment, a portion of the microphone 12 which vibrates during measuring of sound data is substantially parallel to the surface of the frame 2 at the point on the surface of the frame 2. This can help to improve the signal recorded by the microphone 12. However, this is not essential, and the microphone 12 will still record a signal when positioned perpendicular to the surface of the frame 2 at the point on the surface of the frame 2. In an embodiment, such as those shown in FIGS. 3 and 4, the microphone 12 is positioned within a portion of the frame 2 that engages with the teeth 6 of the person 3 when the frame 2 is worn within the mouth of the person 3. The portion of the frame 2 may be a portion of the frame 2 that engages with the incisors or canines of the person 3 when the frame 2 is worn within the mouth of the person 3. The microphone 12 may be located in front of the teeth 6 of the person 3 or behind the teeth 6 of the person 3. In either case, a portion of the frame 2 in which the microphone 12 is located may have increased thickness to accommodate the microphone 12. In an embodiment, the microphone 12 is mounted within a portion of the frame 2 that is within the oral cavity of the person 3 when the frame 2 is worn within the mouth of the person 3. This can more directly expose the microphone 12 to vibrations caused by air flowing through the mouth of the person during breathing. Where the microphone 12 is in a portion of the frame 2 within the oral cavity, the microphone 12 may be behind the teeth of the person 3, as mentioned above. In an alternative embodiment, the microphone 12 is positioned within a portion of the frame 2 that engages with the roof of the mouth of the person 3 when the frame 2 is worn within the mouth of the person 3. This may be desirable if the frame 2 is designed to protect the teeth 6 of the person 3, for example if the device 1 is to be worn while playing contact sports such as rugby or American football, as the rigid microphone 12 is not placed in a portion of the frame 2 that engages with the teeth. It may also be advantageous if the thickness of the portion of the frame 2 that engages with the teeth needs to be minimised.

In an embodiment, a minimum distance between the microphone 12 and a surface of the frame 2 is at most 10 millimetres, optionally at most 5 millimetres, optionally at most 2 millimetres, optionally at most 1 millimetre, optionally at most 0.5 millimetres. Having the microphone 12 closer to the surface of the frame 2 can help to improve the quality of the measured sound data. In an embodiment, the microphone 12 is a microelectromechanical system, MEMS, microphone. This type of microphone is preferred due to its higher tolerance to heat and vibration than other comparable microphones of suitable size. However, in principle any type of microphone may be used that can be manufactured at a sufficiently small size to be positioned within the frame 2. In an embodiment, the microphone 12 comprises a surface that vibrates during measurement of sound data, and a casing. The casing may comprise an entry to allow audio signals to more easily reach the vibrating surface. In an embodiment, the device 1 comprises an amplifier. The amplifier may be connected to the microphone 12 and configured to amplify the signal measured by the microphone 12.

The device further comprises a cavity 14 located within the frame 2 and adjacent to the microphone 12, wherein the cavity 14 does not communicate with the environment surrounding the frame 2. Providing the cavity 14 ensures that the microphone 12 is able to function properly, even where it is encapsulated within the frame 2. Providing the microphone 12 within the frame 2 without the adjacent cavity 14 is likely to degrade the sound data quality to the extent that it is not possible to measure useful sound data. Providing the cavity 14 such that it does not communicate with the environment prevents contaminants, such as moisture or debris, from entering the cavity 14 and/or affecting the performance of the microphone 12. In an embodiment, such as shown in FIG. 4, a first surface of the frame 2 is configured to engage against the mouth and/or teeth 6 when the frame 2 is worn within the mouth of the person 3, and the cavity 14 is located between the microphone 12 and a second surface of the frame 2 that does not engage against the mouth and/or teeth 6 when the frame 2 is worn within the mouth of the person 3. Having the cavity 14 face away from the interior of the mouth, or the teeth of the person, may improve the signal quality detected by the microphone 12. However, adequate signal quality can still be obtained using a configuration such as shown in FIG. 3, where the cavity 14 faces towards the interior of the mouth, or the teeth 6.

In an embodiment, the cavity 14 has a minimum dimension of at least 0.05 millimetres, optionally at least 0.1 millimetres, optionally at least 0.2 millimetres, optionally at least 0.5 millimetres. In an embodiment, the cavity 14 has a maximum dimension of at most 5 millimetres, optionally at most 4 millimetres, optionally at most 3 millimetres, optionally at least 2 millimetres. In an embodiment, the dimensions of the cavity 14 are within the range 0.1 to 4 millimetres, optionally within the range 0.5 to 2 millimetres. Too small a cavity 14 may not allow the microphone 12 to function correctly, while too large a cavity may reduce the strength of the frame 2 or its ability to protect the teeth 6 of the person 3. The cavity 14 may be substantially spherical, or may take other shapes, such as cylindrical. The cavity 14 may have unequal size in different dimensions, for example being smaller in a direction of the thickness of the frame 2 than in directions perpendicular to the thickness of the frame 2. In an embodiment, the dimensions of the cavity 14 are equal to or less than the dimensions of the microphone 12. In an embodiment, a minimum distance between the microphone 12 and an edge of the cavity 14 is at most 0.1 millimetres, optionally at most 0.05 millimetres, optionally at most 0.02 millimetres. In an embodiment, the microphone 12 is exposed to the cavity 14. For example, there may be a direct path, uninterrupted by solid material, connecting the surface of the microphone 12 that vibrates during measuring of sound data with the interior of the cavity 14. Where the microphone 12 comprises a casing with an entry, the cavity 14 may overlap the entry. The cavity 14 may be formed during manufacture of the frame 2. For example, where the frame 2 is formed using multiple layers of material, the cavity 14 may be formed by inserting a spacer between two layers of the frame 2. The spacer may comprise a tube or hollow sphere. Alternatively, where the frame 2 is manufactured using 3D printing, then the cavity 14 may be formed by leaving a gap in the frame 2 during printing.

In an embodiment, the device 1 further comprises a processor 20. In an embodiment, the processor 20 is mounted within the frame 2. For example, in the embodiment of FIG. 5, the electronic components of the device 1 are provided on the circuit board 10, as mentioned above, and the processor 20 is provided on the circuit board 10. The processor 20 may be provided within the frame 2 separately from the other electronic components of the device 1, and be connected to the other electronic components by wires. In an embodiment, a length of a wire carrying sound data between the microphone 12 and the processor 20 is less than 10 centimetres, optionally less than 5 centimetres, optionally less than 2 centimetres, optionally less than 1 centimetre. Reducing the length of wire between the processor 20 and the microphone 12 reduces the potential for noise to be introduced to the sound data, for example from electromagnetic interference. The processor 20 is configured to receive sound data measured by the microphone 12, and determine a fatigue metric representing a level of physical fatigue of the person 3 using the received sound data. The fatigue metric may be calculated using the method of the second aspect of the invention. The fatigue metric may be a numerical value. In an embodiment, the fatigue metric is a rating of perceived exertion as measured on the Borg RPE scale, or the Borg CR-10 scale.

In an embodiment, the device 1 further comprises a light emitter 26. The light emitter 26 may be mounted within the frame 2 and is configured to emit light. The light emitter 26 may comprise a light emitting diode (LED). The light emitter 26 may be mounted within the frame 2 such that light emitted by the light emitter 26 is visible to those around the user of the device 1. For example, the light emitter 26 may be mounted in a front portion of the frame 2, facing away from the teeth of the user. Where the device 1 comprises a light emitter 26, the processor 20 is further configured to control emission of light by the light emitter 26 based on the determined fatigue metric. This can be used to provide a visual indication of the level of fatigue of the user to other around them. For example, in an embodiment, the processor 20 is configured to control the light emitter 26 to emit light when the fatigue metric is above a predetermined threshold. For example, if the fatigue metric is a value on the Borg CR-10 scale, the predetermined threshold may be 7 (out of 10). This can be used to warn bystanders that the user's level of fatigue is too high, and that they should rest or reduce their level of physical activity. Alternatively, the processor 20 may be configured to control the light emitter 26 to emit light of a first colour (for example, green) when the fatigue metric is at or below a predetermined threshold (e.g. 7 out of 10 on the Borg CR-10 scale), and to emit light of a second colour (for example, red) when the fatigue metric is above the predetermined threshold. In such an embodiment, the light emitter 26 could be a bicolour LED, or the light emitter 26 could comprise two LEDs of different colours. Providing two colours can give a clear indication of whether a user is at a safe level of fatigue, or a dangerous level of fatigue. Further divisions could also be made. For example, three different colours, e.g. green, yellow, and red, could be used to indicate low, high, and potentially dangerous exertion levels.

In an embodiment, the device 1 further comprises communication means 22 mounted within the frame 2. The communication means 22 can be used to transmit information between components of the device 1. In an embodiment, the processor 20 is separate from the frame 2, and the communication means 22 is configured to transmit the sound data to the processor 20. In an embodiment, the processor 20 is comprised in a mobile device separate from the device 1. The mobile device may be a mobile phone or other mobile device of the person carrying out the physical activity, or the mobile device may be held by an observer supervising the physical activity of the person. Alternatively, the processor 20 may be comprised in a separate body of the device 1 configured to be worn or carried by the person, for example in a pocket or on a belt or strap. The communication means 22 may be used to transmit information from the device 1 to external devices, for example a computer or mobile device. In an embodiment, the communication means 22 comprises a wireless communication means. An example of such an embodiment is shown in FIG. 6. The device 1 is worn within the mouth of the person 3, and the communication means 22 of the device 1 transmits information wirelessly to a mobile device 30. The mobile device 30 may record and/or analyse information transmitted from the device 1. The wireless communication means 22 may communicate using any suitable wireless communication system or protocol, for example Bluetooth Wi-Fi, or radio.

In an embodiment, the device 1 further comprises a memory module 24 configured to store the measured sound data, and/or store information calculated using the measured sound data. The measured sound data may be recorded in the memory module 24 to be later transmitted using the communication means 22 to another device for analysis. Alternatively, where the device 1 comprises a processor 20, the memory module 24 may only store information calculated using the measured sound data, such as the fatigue metric as a function of time. As a further alternative, the memory module 24 may store both the measured sound data and the information calculated using the measured sound data. This may be advantageous if it is desirable to verify the calculations of the processor 20 at a later time. In embodiments where the device 1 comprises communication means 22, the device 1 may store measured sound data or information calculated using the measured sound data instead of, or in addition to, transmitting the sound data or calculated information. In an embodiment, the memory module 24 has sufficient storage capacity to store measured sound data and/or calculated information for at least 15 minutes of continuous operation of the device 1, optionally at least 30 minutes, optionally at least 1 hour, optionally at least 2 hours.

In an embodiment, the device 1 further comprises a power supply, such as a battery. Alternatively, the device 1 may comprise a power receiver to receive power transmitted to the device 1 wirelessly. Where the device 1 comprises a battery, the batter may have sufficient capacity to allow the device 1 to operate continuously for at least 15 minutes, optionally at least 30 minutes, optionally at least 1 hour, optionally at least 2 hours. The device 1 may further comprise means for charging the battery, such as a wired charging port or inductive wireless charging circuit.

As discussed above, in an embodiment, the components of the device 1 are embedded within the frame 2. The components of the device 1, and the design of the frame 2, are chosen to improve safety and comfort of the person 3 using the device 1. For example, the choice of where in the device 1 to place a circuit board 10 comprising some or all of the electronic components of the device 1 (e.g. the choice between the embodiments shown in FIGS. 1 and 2) may depend on factors such as the preference of the user, the shape of their mouth, and the particular combination of components (e.g. communication means 22, memory module 24) comprised by the specific embodiment used. The components are also chosen so as not to require high voltages or currents to operate, or to generate excessive heat that could be injurious or uncomfortable for the wearer. The mouth of the person 3 can be used to regulate the temperature of the device 1 and prevent damage to the components from overheating, as circulation of air and blood around the mouth will help to regulate the temperature. Nonetheless, the components of the device 1 may be chosen to be able to tolerate higher temperature and humidity than would typically be expected in a person's mouth, in order to reduce the likelihood of failure. In an embodiment, the device 1 can operate continuously for at least 1 hour, optionally 2 hours, optionally 3 hours in a steam-filled environment.

The fatigue metric calculated by the processor 20 in some embodiments of the device 1 can be calculated using a computer-implemented method for determining a fatigue metric representing a level of physical fatigue of a person, according to the second aspect of the invention. The method comprises a step S10 of receiving sound data measured within the mouth of the person. In an embodiment, the method comprises a step of measuring sound data within the mouth of the person prior to receiving the measured sound data. The sound data is measured using a microphone such as that discussed above, and is measured as a function of time. The sound data may be digital sound data or analogue sound data. The sound data measured within the person's mouth includes sound due to inhalation and exhalation, and may include other sounds. The measured sound data may be a continuous measurement within the mouth of the wearer, e.g. where the sound data is digital, sampling of the sound at regular intervals at a suitable sampling rate. The measured sound data may be an amplitude of the sound within the mouth of the person.

The method further comprises processing the sound data to determine at step S12 a breathing rate of the person and determine at step S14 an amplitude of the sound data. The determining of the breathing rate and amplitude may be carried out in any order or simultaneously. The processing may be carried out by a processor located nearby the microphone and within the mouth of the person. Alternatively, the sound data may be received by a processor located outside the mouth of the person for processing. In an embodiment where the measured sound data is analogue data, processing the sound data comprises digitising the measured sound data. In an embodiment, processing the sound data comprises bandpass filtering the sound data to exclude frequencies outside of the range 10 Hz to 2 kHz, optionally outside the range 20 Hz to 1.5 kHz, optionally outside the range 50 Hz to 1 kHz, optionally outside the range 100 Hz to 500 Hz. This filtering helps to exclude components of the sound data which are not derived from the person's breathing, and therefore reduce noise, for example due to sounds produced externally to the person's mouth. Filtering may be performed digitally by processing digitised sound data, or could be performed by processing analogue sound data prior to digitisation using an analogue filter such as an RLC circuit. The breathing rate may be determined from the sound data using any suitable method, for example wavelet analysis, peak finding algorithms, or from a Fourier transform of the sound data as a function of time. In an embodiment, determining the breathing rate and the amplitude comprises calculating a moving average of the breathing rate and/or the amplitude over at least three breaths, optionally at least five breaths, optionally at least ten breaths, taken by the person. Using an average over several breaths can help to smooth fluctuations in the determined data, and enable accurate determination of the breathing rate. Using a moving average enables the processed data to follow changes in the breathing rate and amplitude.

The method further comprises determining the fatigue metric using the breathing rate and the amplitude. The fatigue metric may be a numerical value. In an embodiment, the fatigue metric is a rating of perceived exertion as measured on the Borg RPE scale, or the Borg CR-10 scale. In an embodiment, the fatigue metric is determined using a product of the breathing rate and the amplitude. In such an embodiment, the method further comprises a step S16 of calculating the product of the breathing rate and the amplitude, and a step S18 of determining the fatigue metric from the product. A higher fatigue metric, indicating a higher level of exertion by the person, will be associated with an increased breathing rate and/or increased amplitude of measured sound data relative to the breathing rate or amplitude at rest or at a lower level of exertion. In an embodiment, the sound data, and/or the product of the breathing rate and the amplitude of the sound data is filtered. This may be achieved using bandpass filtering, or lowpass filtering to remove noise. Although filtering can reduce noise, it is not essential to filter the sound data, or the breathing rate or amplitude determined using the sound data, in order to determine the fatigue metric. The raw measured sound data can also be used directly to determine the breathing rate and amplitude and determine the fatigue metric. Further, although in some embodiments the product of the breathing rate and amplitude may be filtered as a function of time to reduce noise, this is also not essential, and the unfiltered product may be used. In an embodiment, processing the sound data comprises obtaining an envelope signal of the sound data and/or the product of the breathing rate and the amplitude as a function of time. In an embodiment, determining the fatigue metric comprises calculating a moving average of the product of the breathing rate and the amplitude. In an embodiment, the moving average is an exponential moving average. An exponential moving average may be used when a moving average of the breathing rate and/or amplitude is calculated, as discussed above. Alternatively, other types of moving average may be used, including an unweighted moving average, or weighted moving averages other than an exponential moving average, such as a linearly weighted moving average.

In an embodiment, determining the fatigue metric comprises normalising the product of amplitude and breathing rate. In an embodiment, the normalisation is performed using the maximum value of the product recorded for the person. In such an embodiment, determining the fatigue metric may comprise recording the value of the product when the first sound data is received and, for each subsequent point in time, comparing the calculated product to the recorded value of the product. If the most recently calculated product is larger than the recorded value, the recorded value is replaced by the most recently calculated product. In such an embodiment, normalising using the maximum value of the product recorded for the person comprises dividing the most recently calculated product by the recorded value. This maximum value (which is recorded as the recorded value) will reflect the point at which the user is perceived to have had the highest level of exertion. As described, the maximum value may be updated whenever the user surpasses the previous maximum value.

Alternatively, other forms of normalisation may be used. In an embodiment, the product may be normalised using multiple previous values of the product. For example, several of the highest values of the product may be recorded, e.g. the two highest values, optionally three, optionally four or more. For each subsequent point in time, the calculated product is compared to each of the recorded values, and the largest recorded value less than the most recently calculated product is replaced with the most recently calculated product. In an embodiment, normalising using the maximum value of the product comprises dividing the product by an average of the recorded values. This approach may help to reduce the effect of outliers on the determined fatigue metric.

The normalisation of the value of the product creates a personalised scale against which any other values of the product can be compared. In an embodiment, the lowest ⅓ of the normalised values represent low levels of perceived exertion, the middle ⅓ of the normalised values represent moderate exertion, and the highest ⅓ represent high exertion. In an embodiment, these thresholds can be changed according to the preferences of the person using the device, or others supervising their activity, for example their coach, or a sport physiologist. These three levels can also be visualised by applying a colour coding scheme, such as that discussed above in relation to the light emitter of the device 1. A multicolour light emitter, such as an LED, can be controlled to emit green light for “low” values of the normalised product, yellow light for “moderate” values, and red light for “high” values. Alternatively, a light emitter may be configured to emit light of a single colour only when the normalised value is within the range representing high exertion. This allows for a quick assessment of the exertion and/or fatigue levels of the person during physical activity (e.g. players on a playing field) without the need of any other equipment. In other embodiments, the normalised values of the product could be mapped to another scale, such as the Borg CR-10 scale. The thresholds used to map the normalised product to the divisions on the scale may be predetermined, or may be selectable by the person, or another supervising the physical activity of the person.

In an embodiment, the method further comprises a step S20 of outputting the determined fatigue metric. The determined fatigue metric may be outputted to a memory to be stored for later retrieval. Alternatively or additionally, the determined fatigue metric may be outputted to a communication means to be transmitted to devices external to the device carrying out the method. In an embodiment, the method may comprise further steps of controlling equipment or devices on the basis of the determined fatigue metric. For example, as described above, a light emitter may be controlled to emit light if the fatigue metric is above a predetermined threshold, or within a predetermined range.

1. A device for measuring fatigue of a person, the device comprising:
a frame configured to be worn within the mouth of the person;
a microphone mounted within the frame and configured to measure sound data; and
a cavity located within the frame and adjacent to the microphone, wherein:
the cavity does not communicate with the environment surrounding the frame.

2. The device of claim 1, wherein:
a first surface of the frame is configured to engage against the mouth and/or teeth when the frame is worn within the mouth of the person; and
the cavity is located between the microphone and a second surface of the frame that does not engage against the mouth and/or teeth when the frame is worn within the mouth of the person.

3. The device of claim 1, wherein the microphone is substantially parallel to a surface of the frame at a point on the surface of the frame closest to the microphone.

4. The device of claim 1, wherein the microphone is mounted within a portion of the frame that is within the oral cavity of the person when the frame is worn within the mouth of the person.

5. The device of claim 4, wherein the microphone is positioned within a portion of the frame that engages with the roof of the mouth of the person when the frame is worn within the mouth of the person.

6. The device of claim 1, wherein the cavity has a minimum dimension of at least 0.05 mm.

7. The device of claim 1, wherein the cavity has a maximum dimension of at most 5 mm.

8. The device of claim 1, wherein a minimum distance between the microphone and an edge of the cavity is at most 0.1 mm.

9. The device of claim 8, wherein the microphone is exposed to the cavity.

10. The device of claim 1, wherein a minimum distance between the microphone and a surface of the frame is at most 10 mm.

11. The device of claim 1, further comprising a processor configured to receive sound data measured by the microphone, and determine a fatigue metric representing a level of physical fatigue of the person using the received sound data.

12. The device of claim 11, further comprising a light emitter mounted within the frame and configured to emit light, wherein the processor is further configured to control emission of light by the light emitter based on the determined fatigue metric.

13. The device of claim 12, wherein the processor is configured to control the light emitter to emit light when the fatigue metric is above a predetermined threshold.

14. The device of claim 12, wherein the processor is configured to control the light emitter to emit light of a first colour when the fatigue metric is at or below a predetermined threshold, and to emit light of a second colour when the fatigue metric is above the predetermined threshold.

15. The device of claim 11, wherein the device further comprises communication means mounted within the frame.

16. The device of claim 15, wherein the communication means comprises a wireless communication means.

17. The device of claim 16, wherein the wireless communication means communicates using at least one of Bluetooth®, Wi-Fi, or radio.

18. The device of claim 15, wherein the processor is separate from the frame, and the communication means is configured to transmit the sound data to the processor.

19. The device of claim 11, wherein the processor is mounted within the frame.

20. The device of claim 19, wherein a length of a wire carrying sound data between the microphone and the processor is less than 10 cm.

21. The device of claim 1, wherein at least a portion of the frame is configured to engage with the teeth of the person.

22. The device of claim 21, wherein the frame is configured to surround one or more of the teeth of the person.

23. The device of claim 1, wherein the frame is a mouthguard.

24. The device of claim 1, wherein the frame is formed from an energy absorbing material, for example ethylene-vinyl acetate, EVA.

25. The device of claim 24, wherein components of the device mounted within the frame are embedded within the energy absorbing material.

26. (canceled)

27. The device of claim 1, wherein the microphone is a microelectromechanical system, MEMS, microphone.

28. The device of claim 1, further comprising a memory module configured to store the measured sound data, and/or store information calculated using the measured sound data.

29. A computer-implemented method for determining a fatigue metric representing a level of physical fatigue of a person, the method comprising receiving sound data measured within the mouth of the person, processing the sound data to determine a breathing rate of the person and an amplitude of the sound data, determining the fatigue metric using the breathing rate and the amplitude.

30. The method of claim 29, wherein the fatigue metric is determined using a product of the breathing rate and the amplitude.

31. The method of claim 30, wherein the sound data, and/or the product of the breathing rate and the amplitude of the sound data is filtered.

32. The method of claim 29, wherein processing the sound data comprises bandpass filtering the sound data to exclude frequencies outside of the range 10 Hz to 2 kHz.

33. The method of claim 30, wherein processing the sound data comprises obtaining an envelope signal of the sound data and/or the product of the breathing rate and the amplitude as a function of time.

34. The method of claim 29, wherein determining the breathing rate and the amplitude comprises calculating a moving average of the breathing rate and/or the amplitude over at least three breaths taken by the person.

35. The method of claim 30, wherein determining the fatigue metric comprises at least one of:
calculating an exponential moving average of the product of the breathing rate and the amplitude; or
normalising the product of the breathing rate and the amplitude.

36. (canceled)
